(19) United States
(12) Patent Application Publication
(10) Pub. No.: US 2015/0283276 A1
(43) Pub. Date: Oct. 8, 2015

(54) Method for Controlling Fungal Plant Pathogens Using a Combination of UV Radiation Followed by Antagonist Application and Dark Period

(71) Applicant: The United States of America, as Represented by the Secretary of Agriculture, Washington, DC (US)

(72) Inventors: Wojciech J. Janisiewicz, Frederick, MD (US); Fumiomi Takeda, Martinsburg, WV (US)

(21) Appl. No.: 14/247,948

(22) Filed: Apr. 8, 2014

(51) Int. Cl. A61L 2/00; A61K 35/66

(52) U.S. Cl. CPC A61L 2/0047; A61K 35/66

(57) ABSTRACT

Strawberries are available year-around from production in the field or from controlled environments (e.g. high and low tunnel culture and greenhouse). Diversity of production conditions results in challenges in controlling diseases before, during, and after harvest. Fungicides, traditionally used to control diseases, have limitations. UV-C irradiation followed by a dark period was used to kill two major pathogens of strawberry, Botrytis cinerea and Colletotrichum acutatum. The UV-C irradiation and dark period was followed by repopulation with beneficial biocontrol microorganisms. The 4 hr dark period prevented activation of a light-dependent UV-C damage repair mechanism in the pathogens. This combination protocol makes it possible to use a lower dose of UV-C for reduction and/or elimination of pathogens. A mobile treatment apparatus was designed to provide the appropriately timed UV-C doses, dark period, and sprayable doses of biocontrol microorganisms. The UV-C dose and repeated exposure did not affect pollen germination or cause chlorophyll degradation in strawberry leaves.

### BACKGROUND OF THE INVENTION

1. Field of the Invention

This invention relates to a method for controlling fungal plant pathogens on strawberries and other crop plants by using a treatment protocol combining UV-C exposure followed by a period of darkness and repopulation with beneficial biocontrol microorganisms. This combination protocol makes it possible to use a lower dose of UV-C for reduction and/or elimination of pathogens. The invention further relates to an apparatus and system to be used for said treatment protocol.

2. Description of the Relevant Art

Strawberry fruit are currently available year round due to the increased production resulting from protective culture conditions as for example in high tunnels before and after the field season. Diseases such as gray mold (cause by Botrytis cinerea), anthracnose (caused by Colletotrichum acutatum) or powdery mildew (caused by Podoshaera aphanis) can cause severe losses by reducing yield and causing fruit decay during production and after harvest, if not controlled from early on in the production cycle (Burlakoti et al. 2013. Intl. J. Fruit Sci. 13:19-29; Carisse et al. 2013. Plant Dis. 97:345-362; Smith, B. J. 2013. Intl. J. Fruit Sci. 13:91-102; Xiao et al. 2001. Plant Dis. 85:901-909). For example, for control of gray mold, it has been determined that control measures must be initiated in the field at the bloom time in order to prevent or reduce infection of flowers because infected flowers can account for approximately 80 percent of fruit decay after harvest (Bulger et al. 1987. Phytopath. 77:1225-1230). Fungicides have been traditionally used for controlling these diseases and are usually applied at regular intervals from the early flowering stage until harvest time (Bulger et al., supra; Wedge et al. 2007. Crop Protect. 26:1449-1458; Wilcox and Seem. 1994. Phytopath. 84:264-270). However, use of fungicides introduces additional problems because a period of time is required between the application of the fungicide and the agricultural workers' reentry to the treated areas, including tunnels, fields, glass houses, warehouses, and other production areas. Fungicide applications can interfere with the harvest, which could be as frequent as every 2-3 days. The search for alternatives to synthetic fungicides is also necessitated by the constant threat of new regulations limiting use of pesticides, especially in protected culture, and by an increasing market demand for fruit free of pesticides (Offner, J. 2013. Retrieved from the Internet: <URL: thepacker.com/fruit-vegetable-news/marketing-profiles/organic/Category-mostly-oblivious-to-economic-swings-186517471.html (Last accessed Aug. 29, 2013); Hanson et al. 2013. Intl. J. Fruit Sci. 13:73-77). Integrated pest management and biological control in both protected and open field productions have made significant progress during past decades; however, more is needed to reduce losses and make this system more profitable (Pickett Pottorff and Panter. 2009. HortTech. 19:61-65).

Biological control approaches to control strawberry diseases have been tried with considerable success; however, by themselves, they are not as effective as fungicide treatments. Further, no commercial biocontrol product has as yet been used in strawberry production despite farmers' positive attitudes to using biocontrol agents in strawberry fruit production in such main growing areas as, for example, Germany, Italy and Israel (Moser et al. 2008. Biol. Control 47:125-132; Bhatt and Vaughan. 1962. Plant Dis. Rep. 46:342-345; Karabulut et al. 2004. Biocontrol Sci. Technol. 14:513-521; Lima et al. 1997. Postharvest Biol. Technol. 10:169-178; Sylla et al. 2013. Crop Protection 51:40-47; Peng et al. 1992. Can. J. Plant Pathol. 14:117-188; Pertot et al. 2008. Crop Protection 27:622-631; Xu et al. 2010. Biocontrol Sci. Technol. 20:359-373).

In comparison, combining biological control with comparable physical or chemical treatments has been very successful for increasing the effectiveness of disease control on fruit, in particular, on fruit after harvest (see review by Janisiewicz and Conway. 2011. Stewart Postharvest Rev. 9(1):1-16).

UV-C (ultraviolet radiation with wavelengths between 200 and 290 nm with the peak between 240 and 265 nm) has been used to kill microorganisms in various systems including: sterilization of air in hospitals, sterilization of water in treatment plants, and to some extent, in agriculture and in the food industry (Beggs et al. 2006. Aerosol Sci. 37:885-902; Gardner and Shama. 2000. J. Food Protect. 63:63-73; Hijnen et al. 2006. Water Res. 40:3-20). Postharvest treatment of potatoes, carrots, tomatoes, bell peppers, table grapes, strawberries, apples, peaches and citrus fruit with UV-C induced resistance to decay-causing fungi has resulted in reduction of decay (Adrian et al. 2000. J. Agric. Food Chem. 48:6103-6105; Chalutz et al. 1992. J. Phytochem. Phytobiol. 15:367-374; Charles et al. 1999. Phytopath. 89 (Suppl):S14; Droby et al. 1993. Plant Path. 42:418-424; Stevens et al. 1998. Crop Prot. 17:75-84; Wilson et al. 1994. Plant Dis. 78:837-844; Mercier et al. 2000. Phytopath. 90:981-986; Nigro et al. 1998. Postharvest Biol. Technol. 13:171-181; Nigro et al. 2000. J. Plant Pathol. 82:29-37). Storage decay has been significantly reduced by treatment of harvested strawberries with UV-C alone or with pulsed white light and heat (Marquenie et al. 2003. Postharvest Biol. Technol. 28:455-461; Nigro et al. 2000, supra). The combination of UV-C and heat made possible a reduction in the intensity of the treatments for inactivation of Botrytis cinerea and Monilinia fructicola conidia (Marquenie et al. 2002b. J. Food Microbiol. 74:27-35). Treatment of apple slices with UV-C was more effective in controlling foodborne pathogens such as Escherichia coli, Listeria innocua or Salmonella enterica than conventional treatment with sodium chloride. An added advantage was that there were no negative effects on quality of the slices (Graça et al. 2013. Postharvest Biol. Technol. 85:1-7). Treatment of leafy vegetables such as spinach or lettuce with UV-C has also been effective in reducing populations of foodborne pathogens and other bacterial microflora. However, softening of the tissue may occur when higher doses are used to ensure reduction of a higher percentage of the pathogenic population (Escolana et al. 2010. Postharvest Biol. Technol. 56:223-231; Allende et al. 2006. Food Microbiol. 23:241-249). Ultraviolet irradiation of Botrytis fabae, a fungal pathogen of beans (Vicia faba), reduced conidia infectivity more rapidly than their viability, as determined by growth on agar media (Buxton et al. 1957. J. Gen. Microbiol. 16:764-773). An exposure to light after irradiation reduced the effect of ultraviolet treatment (Last and Buxton. 1955. Nature (London) 176:655). Despite these considerable potential benefits of using UV-C for controlling various diseases, this approach has only been sporadically used under commercial conditions. The damaging effect to plants at doses required to kill a substantial part of the pathogen population and the limited and often variable affect of UV-C on induction of resistance in fruit appears to have contributed to the lack of commercial implementation thus far.

Irradiation of strawberry plants with UV-C can kill a significant part of the non-pathogenic microbial population on the plant surface, in addition to the pathogen, thus creating a microbial vacuum. Because the natural microbial population is diminished or absent, there is a lack of competition when newly-arriving, airborne conidia of pathogens occur, giving pathogens a colonization advantage. Thus, an essential part of any practical treatment requires combining UV-C treatment with good colonizers of strawberry plants, in particular, colonizers of those plant parts vulnerable to infection by the pathogen. Using colonizers that are antagonistic to strawberry pathogens would considerably improve efficacy and reliability of the system.

Various biocontrol methods and formulations for effective control of pathogenic fungi are known in the art; however, there still remains a need for biocontrol strategies which are not only effective, but which also ensure the quality of the agricultural food crops that are being protected.

### SUMMARY OF THE INVENTION

We have discovered that a biocontrol strategy combining treating plants with a low dose of UV-C (low dose/short time period) followed by a period of darkness dramatically reduces survival and infection by plant pathogens and has a limited or no effect on plant growth, pollen germination, or quality of fruit or vegetables produced by said plant.

In accordance with this discovery, it is an object of the invention to provide a treatment protocol using a combination of a UV-C dose, period of darkness, and administration of biocontrol agent.

It is further object of the invention to provide a non-chemical treatment to control/reduce pathogen growth on living plants which affects the pathogen without causing any permanent negative effect on the crop plant, in particular, without having a negative effect on the normal growth and development of the plant.

It is another object of the invention to provide a biological control method which allows growers to control pathogen growth without affecting the normal growth and development of the crop plants, thus significantly reducing crop losses.

It is an additional object of the invention to provide an alternative to fungicide control methods, which includes, in particular, a combination of a UV-C dose, period of darkness, and administration of biocontrol agent.

It is still further object of the invention to provide a method of treatment for controlling pathogens on agricultural crops that will be harvested within the next few days after the treatment.

It is another object of the invention to provide a treatment method for controlling pathogens on agricultural crops which requires no reentry period after application of the treatment.

It is another object of the invention to provide a treatment method for controlling pathogens on agricultural crops without having a negative effect on pollen germination and fruit set or causing chlorophyll degradation of crop plants.

Other objects and advantages of this invention will become readily apparent from the ensuing description.

### BRIEF DESCRIPTION OF THE DRAWINGS

The patent or application file contains at least one drawing executed in color. Copies of this patent or patent application publication with color drawing(s) will be provided by the U.S. Patent and Trademark Office upon request and payment of the necessary fee.

FIG. 1 shows the effect of a dark incubation period (0, 1, 2, 3, 4, 5 and 6 hr) on germination of Botrytis cinerea conidia irradiated with UV-C (254 nm) for various times (0, 30 and 60 sec; 3 replicate plates shown for each treatment). Plates were photographed 72 h after irradiation.

FIG. 2 shows the effect of a dark incubation period (0, 1, 2, 3, 4, 5 and 6 hr) on germination of Colletotrichum acutatum conidia irradiated with UV-C (254 nm) for various times (0, 15 and 30 sec; 3 replicate plates shown for each treatment). Plates were photographed 72 h after irradiation.

FIG. 3 depicts the progressive damage of chlorophyll in strawberry leaves over an 11 day period (on Day 0, 1, 2, 3, 6 and 11) after exposure to UV-C for up to 6 hr (i.e., for 0, 2, 4 and 6 hr).

FIG. 4 depicts the lack of chlorophyll damage in strawberry leaves after exposure to UV-C for 0, 30 and 60 sec two times a week for seven weeks in the high tunnel culture. Numbers in red boxes indicate intensity of fluorescence.

FIGS. 5A and 5B show pollen tubes growing in sucrose/boric acid medium. In FIG. 5A, pollen tubes are from non-irradiated pollen and in FIG. 5B, pollen tubes are from pollen irradiated with UV-C (254 nm) for 60 sec and incubated in the dark for 4 hr. Pollen has been stained with lactophenol cotton blue.

FIGS. 6A and 6B show pollen tubes growing from pollen after UV-C (254 nm) irradiation for 60 sec and incubation in the dark for 4 hr. In FIG. 5A, pollen tubes are in sucrose/boric acid medium and in FIG. 5B, pollen tubes are shown within a style after the pollen was deposited on the stigma of the pistil of an emasculated strawberry flower. The pollen has been stained with aniline blue and viewed with fluorescent microscopy.

FIG. 7 depicts infectivity of B. cinerea conidia after exposure to UV-C for various times. Wounds on the apple were inoculated with 25 μL of conidial suspension (10⁴ conidia/mL) and incubated at 22° C. for 5 days.

FIG. 8 depicts the recovery of Metschnikowia pulcherrima (FMB-24H-2) populations from strawberry anthers and emasculated flowers misted with the antagonist and incubation at 22° C. for 24, 48 and 72 hr. The applied inoculum was allowed to air dry before the first recovery (time 0 hours).

FIG. 9 depicts the recovery of Aureobasidium pullulans (ST1-C9) populations from strawberry anthers and emasculated flowers misted with the antagonist and incubated at 22° C. for 24, 48 and 72 hr. The applied inoculum was allowed to air dry before the first recovery (time 0 hours).

FIG. 10 shows a top view of the preferred embodiment of the plant treatment apparatus.

FIG. 11 shows a partial sectional side view of the treatment module 12 along the section line XI shown in FIG. 10.

### DETAILED DESCRIPTION OF THE INVENTION

We have designed a treatment protocol for the control of major diseases of strawberry in high tunnel culture. In this study, we determined the susceptibility of B. cinerea and C. acutatum conidia to UV-C (254 nm) irradiation, the effect of inhibition of the DNA UV repair mechanism on viability and infectivity of the conidia, the effect of UV irradiation on viability and tube growth of strawberry pollen, and the potential damaging side effects of the UV-C doses to the strawberry photosynthesis system.

We have demonstrated that incubating B. cinerea and C. acutatum conidia in the dark immediately after UV-C exposure on agar plates in the dark resulted in dramatic increase in kill over those incubated in continuous light. After 60 sec irradiation at 206 μW/m², resulting in 0.001236 J/cm², no colonies developed on agar plates kept for 4 hr in dark after irradiation. This effect is more dramatic than that reported for Botrytis fabae where after 60 sec of UV-C exposure at intensity of 173 μW/m² and a dark period of 7 hr, almost one third of the conidia produced lesions on bean leaves (Buxton et al., supra). Conidia of C. acutatum were much more vulnerable to UV-C treatment then B. cinerea and only a few survived 30 sec exposure. Thus, our subsequent efforts were focused on B. cinerea. Powdery mildew of strawberry, caused by Podosphaera aphanis, could also be controlled with the UV-C treatment regime developed for control of B. cinerea because the mycelium of P. aphanis and other powdery mildew fungi, including Erisiphe graminis and other fungi that reside mainly on plant surfaces, such as for example, Leveillula taurica (previously Erysiphe taurica) on tomato, Oidium neolycopersicum on tomato, Podosphaera xanthii (previously Sphaerotheca fuliginea) on cucumbers and squash, Oidium dianthi on carnations, Podosphaera pannosa var. rosae (previously Sphaerotheca pannosa var. rosae) on roses, Podosphaera fusca on melon and cucurbits, Microsphaera syringae on lilacs and Phyllactinia corylea on trees and shrubs. Amounts of UV-C exposure can be adjusted depending on the sensitivity of the various species of fungi being treated.

Lammertyn et al. (2003. Postharvest Biol. Technol. 30:195-2007) observed sepal dehydration and subsequent browning on harvested strawberries irradiated with doses higher than 0.1 J/cm², a dose that did not provide adequate control. To improve control they combined UV-C irradiation with heat treatment and white light pulses (Marquenie et al. 2002a. Int. J. Food Microbiol. 73:191-200; Marquenie et al. 2003, supra); however, the results were still not satisfactory. Relying solely on treatment(s) after harvest for controlling strawberry decay is only partially effective as most of the infections resulting in fruit decay originate in the field during bloom (Bulgler et al., supra). Thus, the control treatments need to be applied as early as the onset of bloom and need to be continued until harvest. In addition, considering the observations of UV-C damage to sepals, it has been important to determine if the level of UV-C treatment required for killing B. cinerea had any negative effect on photosynthetic apparatus, which in turn may not only effect plant growth and fruit yield, but also appearance of strawberry fruit, and in particular, sepals.

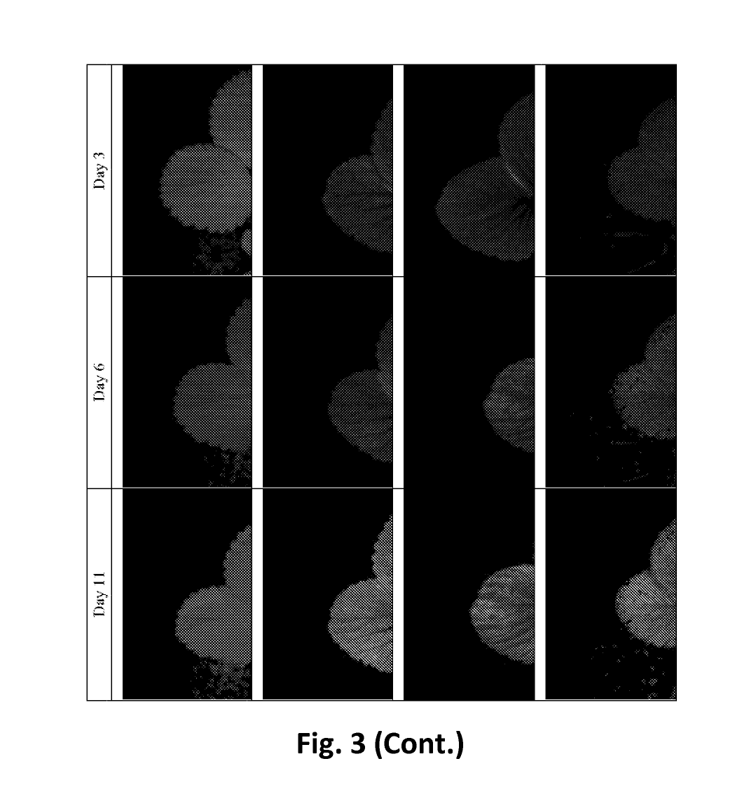

Maintaining a dark environment for 4 hr after UV-C treatment prevented light activation of the of the UV-C repair mechanism (Essen and Klar. 2006. Cell. Mol. Life Sci. 63: 1266-1277; Beggs, C. B. 2002. Photochem. Photobiol. Sci. 1:431-437; Beggs et al. 2006, supra) and thus made it possible to reduce the UV-C dose required for killing B. cinerea to a lowered dose of 0.001236 J/cm², a dose much below the dose levels that were damaging to sepals as reported by Lammertyn (supra). Even after exposing strawberry plants to this dose twice a week for seven weeks, we did not observe any chlorophyll degradation in leaves (FIG. 4) or in sepals. In addition, there were no negative effects of this UV-C dose on pollen germination, tube growth, and length of the tube in the synthetic medium, or on germination on stigma and tube growth in style. Thus, the lack of the negative effects indicates the usefulness of this UV-C approach in controlling diseases of strawberry as well as other fruit crops, flowering and foliage ornamental crops, and vegetables grown in a protective environment (e.g., cucumbers and tomatoes) where the fungi (for example, fungi which cause powdery mildew) reside on the aerial plant surfaces. Irradiation of dry conidia of B. cinerea at this dose (0.001236 J/cm²) resulted in survival of some conidia; however, the irradiated dry conidia were not able to cause fruit infection when tested in a very sensitive assay on mature apples. This observed loss of infectivity prior to loss of viability after UV-C irradiation is in agreement with earlier reports with other plant pathogens (Buxton et al., supra; Moseman and Greeley. 1966. Phytopath. 56:1357-1360).

UV-C treatment of plants to control diseases is a very attractive alternative to synthetic fungicides because it does not leave any residue and does not require a reentry period after application, which can be a significant problem, for example, during harvesting, especially in closed environment cultures. In addition, UV-C treatment may induce resistance in plants, which may, indirectly, improve control of various pre and postharvest pathogens (Nigro et al. 1998, 2000, supra; Petit et al. 2009. J. Exp. Botany 60:1155-1162; Wilson et al., supra).

It is inevitable, that in addition to plant pathogens, other plant surface microflora will be killed by UV-C treatment. Results from limited studies indicate that after initial decline, populations recover rather quickly and even increase beyond the original levels, presumably because of the release of nutrients from plant cells during UV-C irradiation (Nigro et al. 1998, supra; Sztejnberg and Blakeman. 1973. Physiol. Plant Pathol. 3:443-451). However, the population recoveries may not be fast enough to prevent development of iatrogenic diseases (Griffiths, E. 1981. Ann. Rev. Phytopathol. 19:119-182) and composition of the recovered microflora may or may not be favorable to the pathogen, exposing the system to considerable potential variations. In order to reduce this variation, we envision the application of biocontrol agents that can efficiently colonize plant parts most vulnerable to infection by B. cinerea, such as flowers and fruit, immediately after UV-C treatment. Both, UV-C and biocontrol treatments with antagonists do not require reentry period and are acceptable organic treatments, which are very much needed for the rapidly expanding organic market.

As used herein “in amounts effective”, “an amount effective” or “an effective amount” refer to the administered amounts of the components of the antifungal treatment protocol, i.e., UV-C exposure, time period of darkness, dosage of beneficial biocontrol microorganisms, wherein the effect of the administration acts to control pathogens, to reduce populations of pathogens, to reduce fungal contamination of agricultural commodities or is effective to obtain a reduction in the level of disease, as measured by fungal growth or the symptoms associated with fungal growth, relative to that occurring in an untreated control under suitable conditions of treatment. In cases where the composition of the invention is applied prophylactically, use of these terms means that the disease is prevented at a significant level relative to untreated controls. The actual rate and amount of application will vary depending on the fungal organism being controlled, the point in its growth phase that treatment is commenced, the substrate being treated and other environmental factors. In the bioassays conducted as described in Example 2 below, for example, the treatment of the invention was shown to be effective in vitro against the germinating conidia of several pathogenic fungi. The effective amount of the components of the antifungal treatment protocol, i.e., UV-C exposure, time period of darkness, dosage of beneficial biocontrol microorganisms is an amount sufficient to prevent or treat the adverse effects of a fungal-induced infection, disease and/or condition. The particular dose regimen will be dependent upon a plurality of factors, such as the species, the size, and the developmental stage of plant or crop being treated, the target fungal species, the severity of infection, the method of application, etc. For example, the amounts of exposure to UV-C can be adjusted depending on the sensitivity of the fungi being treated. Upon taking these factors into account, actual dose level and regimen could be readily determined by the person of ordinary skill in the art.

By “reduce” or other forms of the word, such as “reducing” or “reduction, meant lowering of an event or characteristic (e.g., fungal growth or survival). It is understood that this is typically in relation to some standard or expected value, in other words it is relative, but that it is not always necessary for the standard or relative value to be referred to. For example, “reduces fungal population growth” means decreasing the population relative to a standard or a control.

By “prevent” or other forms of the word, such as preventing” or “prevention,” is meant to stop a particular event or characteristic, to stabilize or delay the development or progression of a particular event or characteristic, or to minimize the chances that a particular event or characteristic will occur.

By “treat” or other forms of the word, such as “treated” or “treatment,” is meant to administer a composition or to perform a method in order to reduce, prevent, inhibit, breakdown, or eliminate a particular characteristic or event (e.g, fungal growth or survival). The term “to control” is used synonymously with the term “to treat”

The term “UV-C light” or “UV-C radiation” refers to ultraviolet light (or radiation) having a wavelength of between 200 and 290 nm. UV-C light, having the peak wavelength of 254 nm is the preferred wavelength for the treatment protocol of the invention. This definition encompasses end-point wavelengths of 240-265 nm, or values or ranges in between the end-points, such as about 254 nm.

“Biocontrol agent” or “biological control agent” is used to describe a naturally occurring living organism, such as fungi and bacteria, that can be screened to control plant pathogens and pests. Here, the biocontrol agent inhibits the pathogenic organism's growth and development. The efficiency of the biocontrol agent can be increased by altering the environment to favor the development of the biocontrol agent—allowing them to express the mechanisms of inhibition including competition for limiting nutrients and space resulting in the restriction of disease development by the pathogen.

“Live plants” or “living plants” is used herein to refer to plants of any growth stage, ranging from seedling stages to mature plants.

“Parts of a plant” refer herein to parts of the live plants, which are not removed from the plants. For example, the stem or lower side of the leaves are parts of a whole plant. Also, a region of a plant is a part of a plant, as for example a lower part of a plant.

A “plurality of plants” are plants grown in proximity of each other, e.g., side by side in rows or in a field.

“Aerial surfaces” or “aerial plant parts” is the surface of the plant above ground, especially the foliage, stems, flowers, and developing fruit.

“Pathogen” or “plant pathogen” refers herein to microorganisms, such as fungi, bacteria, mycoplasmas and viruses, which are able to cause diseases (e.g., seen as symptoms) on live plants, i.e. on host plants. Especially referred to are pathogens which are present during at least one part of their life-cycle on the surface of one or more of the aerial parts of plants.

“Contact” or “contacting” in the context of UV-C light refers to the shining of the light onto a surface and therefore the exposure of the surface to the UV-C light. “Contacting with” and “exposure to” are herein used interchangeably.

“Controlling pathogen growth” refers to the reduction of the total amount of one or more pathogens on the plant or on one or more plant parts. Reduction can be due to parts of the pathogen being killed, damaged, or affected in their growth rate, reproduction and/or spread.

### EXAMPLES

Having now generally described this invention, the same will be better understood by reference to certain specific examples, which are included herein only to further illustrate the invention and are not intended to limit the scope of the invention as defined by the claims.

### Example 1

### Pathogens

Botrytis cinerea (isolate J4) was originally isolated from decayed apple and was used in various earlier studies on pome fruits and strawberries because it is one of the most aggressive isolates in our collection. Colletotrichum acutatum was isolated from a strawberry and was kindly provided by Dr. Barbara Smith from USDA-ARS Thad Cochran Southern Horticultural Lab, in Poplarville, Miss. Conidia of B. cinerea were collected from 10-14 day-old cultures grown on potato dextrose agar (PDA) by a hand-held vacuum powered cyclone spore collector (Geoff Harms, Physics Laboratory, University of Minnesota, St. Paul, Minn.), resuspended in sterile distilled water (SDW), sonicated for 60 sec, vortexed, and adjusted to desired concentrations with hemacytometer. Conidia of C. acutatum were collected from 10-14 day-old cultures grown on oatmeal agar (OMA) with inoculation loop, suspended in SDW, sonicated for 60 sec, vortexed, and adjusted to desired concentration with hemacytometer.

In addition to B. cinerea and C. acutatum, other fungi, e.g., P. aphans, E. graminis, B. fabae and other fungi which cause powdery mildew and/or reside on the surface of agricultural plants and products, such as for example, Leveillula taurica (previously Erysiphe taurica) on tomato, Oidium neolycopersicum on tomato, Podosphaera xanthii (previously Sphaerotheca fuliginea) on cucumbers and squash, Oidium dianthi on carnations, Podosphaera pannosa var. rosae (previously Sphaerotheca pannosa var. rosae) on roses, Podosphaera fusca on melon and cucurbits, Microsphaera syringae on lilacs and Phyllactinia corylea on trees and shrubs, can be treated with our treatment protocol.

### Example 2

### UV-C Irradiation of Conidia

All UV-C irradiation was conducted with lamps having a peak emission at 254 nm (model TUV PL-L 55 Watt; Phillips North America Corp. Andover, Mass.). The lamp was mounted on a frame that allowed for the adjustment of a distance to the targeted irradiation surface (plates or plants) to 30 cm. This distance was selected because it reflects a distance in the future commercial irradiation apparatus. The frame was enclosed to prevent any light penetration of the enclosure. UV-C is applied in the dark, i.e., there is darkness all around the plants except for the UV-C source. UV light was turned on at least 10 minutes prior to irradiation to ensure consistent intensity levels. The irradiation intensity at the distance of 30 cm was 0.206 W/m² (20.6 μW/cm²). Thus, the 60 sec illumination corresponded to 12.36 J/m² (0.001236 J/cm²). Irradiance was measure with a calibrated spectrometer (StellarNet, Inc. EPP2000, Tampa, Fla.).

Conidial suspension adjusted to 1×10⁴ conidia/mL was vortexed thoroughly and 25 μl was deposited onto 5 cm PDA plate. Plates were placed on a tray and after the liquid was absorbed by the medium (approximately 15-20 min) the lids from the plates were removed and the tray was placed under UV-C light at a distance of 30 cm. After exposure to UV-C for the predetermined time the tray was removed, the lids were put back on the plates, the plates were sealed with Parafilm, and either placed immediately in light or incubated in dark for various periods of time before exposing to continuous light at 25° C.

Colonies arising from B. cinera conidia were counted for the first time after 48 hours, when they became visible, and again after 72 hours, when they were more defined and new colonies developed. No additional colonies were observed after longer incubation. Colonies arising from C. acutatum were counted for the first time after 24 hours and again after 48 hrs, to add any new colonies that may have developed. No additional colonies developed after this time.

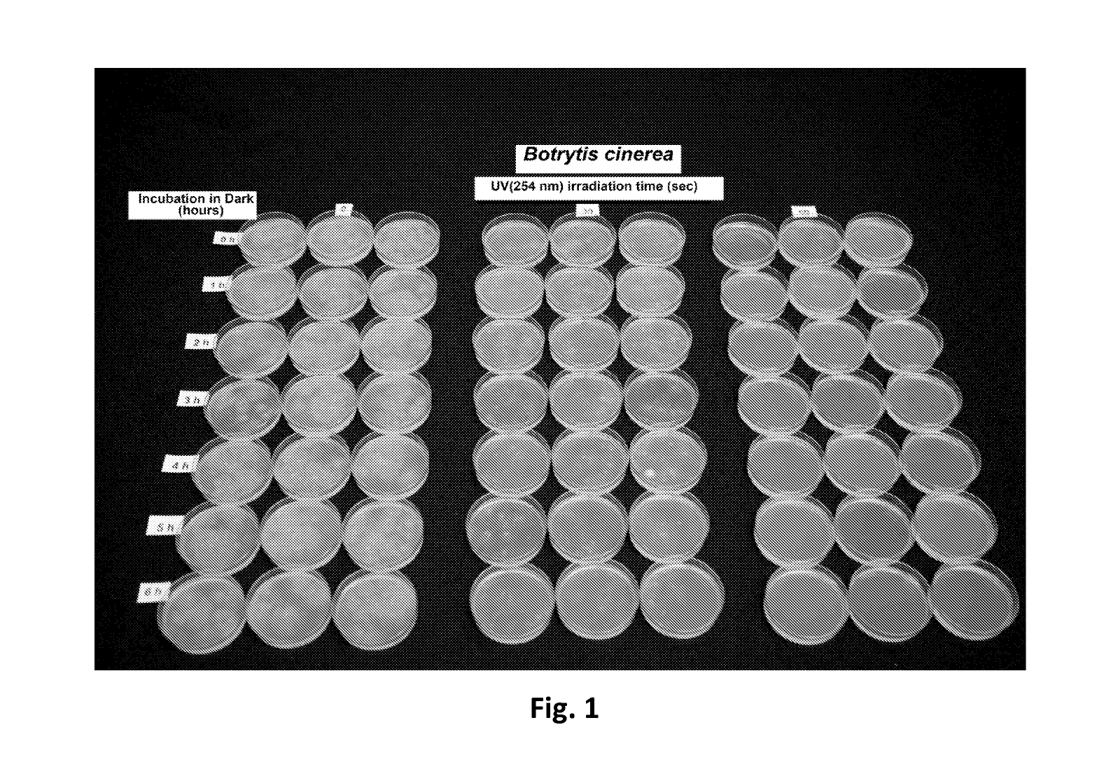

No conidia of B. cinerea germinated after 24 hr incubation, and after 48 hr small, distinct colonies appeared on PDA. Irradiation of the B. cinerea conidia with UV-C for 60 sec resulted in reduction of germinating conidia and development of the colonies from 35 to 13 when conidia were exposed to continuous incandescent light immediately following irradiation (Table 1). However, when irradiated conidia, from the same experimental batch, were kept in dark immediately after the irradiation, the germination and development of the colonies was reduced further as the darkness period increased for up to 4 hr (Table 1, FIG. 1). At this point no colonies developed after moving to continuous incandescent light.

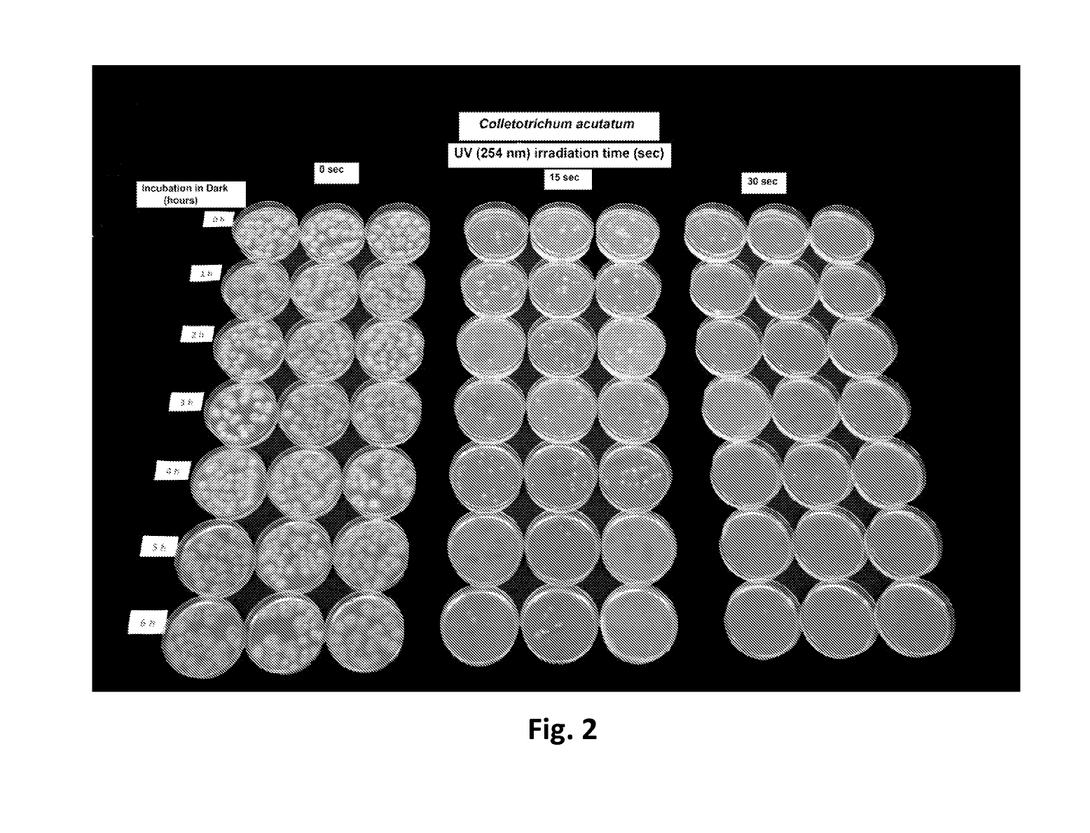

Non-irradiated conidia of C. acutatum germinated and produced distinct colonies after 24 hr incubation, while no colony development or germination was observed on conidia irradiated with UV-C for 15 or 30 sec regardless of the light conditions after the irradiation (Table 2). However, after 48 hr incubation some colonies appeared on plates irradiated for 15 sec. Two hours of dark period after 30 sec irradiation with UV-C was sufficient to prevent any colony development or conidia germination (Table 2, FIG. 2).

TABLE 1
Effect of dark incubation period on germination of
Botrytis cinerea conidia after UV-C (254 nm) exposure.
Germinated conidia (CFU/plate)

| | 24 hr | | | 48 hr | | |
|---|---|---|---|---|---|---|
| Incubation in | UV exposure (sec) | | | UV exposure (sec) | | |
| dark (hr) | 0 | 30 | 60 | 0 | 30 | 60 |
| 0 | 0 | 0 | 0 | 35 (±1.8)* | 21 (±2.9) | 13 (±1.9) |
| 1 | 0 | 0 | 0 | 22 (±1.5) | 20 (±1.0) | 5 (±2.1) |
| 2 | 0 | 0 | 0 | 22 (±2.1) | 18 (±1.8) | 7 (±0.9) |
| 3 | 0 | 0 | 0 | 24 (±1.3) | 16 (±1.9) | 1 (±0.3) |
| 4 | 0 | 0 | 0 | 20 (±3.2) | 13 (±1.2) | 0 |
| 5 | 0 | 0 | 0 | 13 (±3.5) | 25 (±1.5) | 0 |
| 6 | 0 | 0 | 0 | 21 (±3.1) | 11 (±1.3) | 0 |

*Standard error of the mean of three replicates.

TABLE 2
Effect of dark incubation period on germination of
Colletotrichum acutatum conidia after UV-C (254 nm) exposure.
Germinated conidia (CFU/plate)

| | 24 hr | | | 48 hr | | |
|---|---|---|---|---|---|---|
| Incubation in | UV exposure (sec) | | | UV exposure (sec) | | |
| dark (hr) | 0 | 15 | 30 | 0 | 15 | 30 |
| 0 | 52 (±2.3)* | 0 | 0 | 58 (±2.0) | 13 (±5.8) | 0 (±0.3) |
| 1 | 55 (±5.7) | 0 | 0 | 61 (±3.6) | 9 (±0.7) | 1 (±0.9) |
| 2 | 50 (±2.3) | 0 | 0 | 54 (±2.4) | 10 (±5.3) | 0 |
| 3 | 47 (±3.9) | 0 | 0 | 54 (±5.4) | 5 (±1.2) | 0 |
| 4 | 48 (±5.2) | 0 | 0 | 54 (±6.1) | 10 (±3.0) | 0 |
| 5 | 57 (±4.9) | 0 | 0 | 61 (±5.6) | 1 (±0.6) | 0 |
| 6 | 47 (±4.2) | 0 | 0 | 53 (±1.2) | 1 (±0.9) | 0 |

*Standard error of the mean of three replicates.

### Example 3

### Effects of UV-C on Strawberry Pollen Viability, Pollen Germination, Pollen Tube Growth, and Leaf Chlorophyll

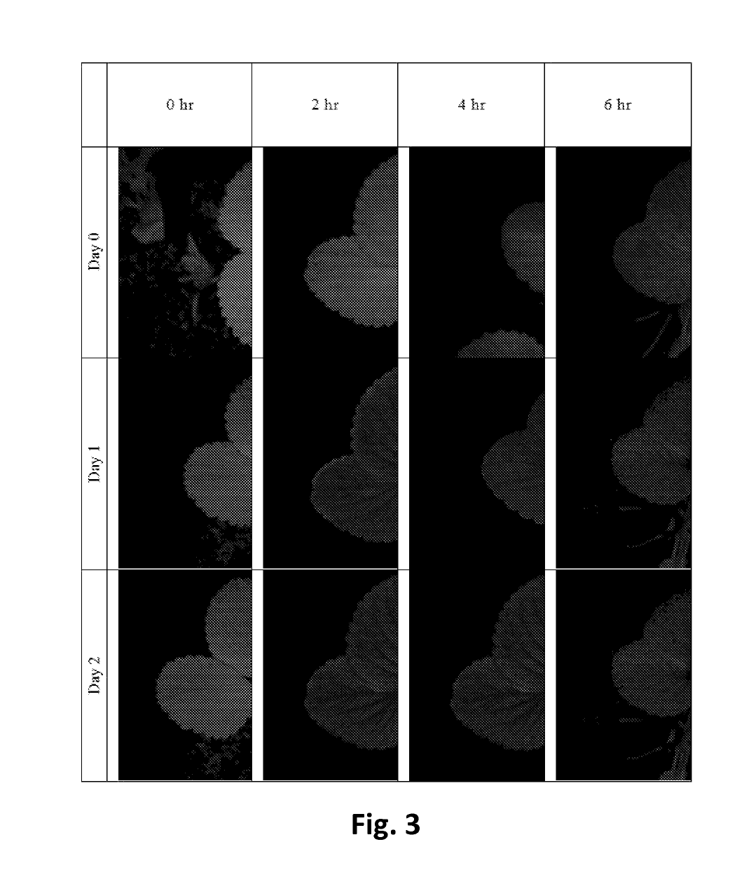

No chlorophyll degradation was observed in leaves of strawberry plants irradiated with UV-C for 30 or 60 sec twice a week for seven weeks (FIG. 4). Significant chlorophyll damage was demonstrated in plants irradiated a single time for 2, 4 or 6 hr, and the damage progressed as the time between irradiation and sampling increased up to 11 days, the duration of the experiment (FIG. 3).

Fully-opened flowers with bright yellow anthers were collected from multiple plants in greenhouse culture. The anthers were removed using sterile forceps and placed in a petri plate (10-cm) overnight to promote dehiscence. The following day the plate was agitated to release pollen from the anthers. The anthers were removed and the pollen collected from all flowers was mixed to create a homogenous blend. The pollen was divided among multiple 5-cm plates to accommodate the number of treatments and replicates.

Lids from the 5-cm plates containing pollen were removed and placed next to the opened plates with the inside surface facing the UV-C bulb at a distance of 30 cm, and irradiated for the designated time. After UV-C exposure the plates were either placed immediately in light or in plastic boxes, covered with black plastic, and incubated in total darkness at 22° C. for up to 6 h. Plates were removed in hourly intervals and the pollen was stained and spread on glass microscope slides to determine viability. Germination and tube growth were determined in pollen growth medium. Also, pollen from 4 h dark incubation was used for pollination of emasculated strawberry flowers.

Viability of both control and UV-C exposed pollen was assessed by fluorescent staining with 10 μM H₂DCF-DA (Sigma, St. Louis, Mo.) in DMSO. Pollen was collected from plates with an inoculation loop, moved to a vial containing 1 mL of the stain and then incubated for 10 min in darkness. The stain was removed through centrifugation at 13,400 rpm for 60 sec. The supernatant was discarded and the pollen was resuspended in 500 μL sterile distilled water (SDW) to wash any remaining stain, centrifuged again (13,400 rpm) for 60 sec and resuspended in 100 μL SDW and examined microscopically (Zeiss Axiophot) for viability. Several random view fields were used to count 100 pollen grains. Grains were rated as viable if they fluoresced and the count was compared to the examination of the same view under bright field illumination.

Irradiation of strawberry pollen with UV-C for 60 sec had no negative effect on pollen viability as determined by fluorescent staining with H₂DCF-DA and concurrent count with the light microscope. The viability was above 70% for the non-irradiated and more than 90% for irradiated pollen (Table 3).

TABLE 3
Effect of UV-C (254 nm) exposure on pollen viability.

| | | |
|---|---|---|
| UV Exposure | Pollen count | Viability |
| (sec) | ViableTotal | (%) |

| | | | |
|---|---|---|---|
| 0 | 78 | 107 | 72.9 |
| 60 | 99 | 103 | 96.1 |

A 100 μL drop of a pollen germination solution (10% sucrose and 0.01% H₃BO₃) was added to the pollen-coated microscope slides. The slides were incubated in Petri plates with moistened filter paper overnight at 25° C. Then the pollen was stained with lactophenol cotton blue and observed microscopically. Several random view fields were examined and 100 pollen grains per replicate were rated on a 4-point scale; 1=not germinated, 2=pollen tube length <2× pollen diameter, 3=pollen tube length 2×-4× pollen diameter, 4=pollen tube length >4× pollen diameter. There were at least three replicates per treatment. Also, the pollen tube length was measured using the “polyline” tool in the DP2-BSW software for the Olympus DP71 microscope digital camera (Olympus America, Inc. Center Valley, Pa.). One hundred pollen tubes were measured per replication and average pollen tube length and standard error of the means were calculated from these values.

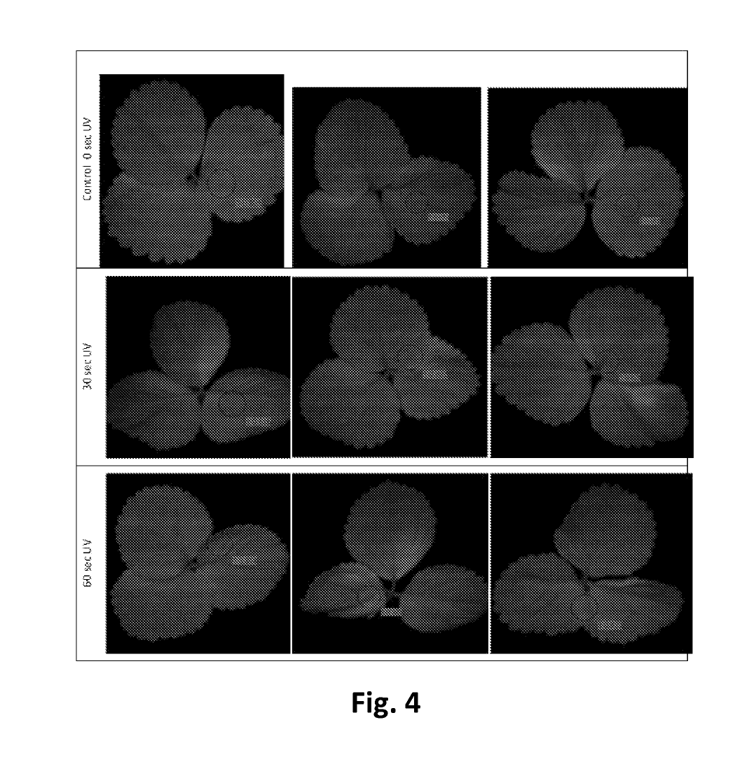

Irradiation of strawberry pollen with UV-C for 60 sec had no negative effect on pollen germination. Different stages of the pollen germination also appeared not to be affected (Table 4). Irradiation of strawberry pollen with UV-C for 60 sec had no negative effect on average pollen tube length in pollen germination medium (Table 5, FIG. 5). Although there were significant variations among replications within a treatment, the lengths of germ tubes in a given replication were consistent as indicated by low standard error of means.

TABLE 4
Effect of UV-C (254 nm) exposure on germination of pollen*.

| UV | | | | | | | |
|---|---|---|---|---|---|---|---|
| exposure | | Rating** | | | | Germination | Average |
| (sec) | Replicate | 1 | 2 | 3 | 4 | (%) | germination |
| 0 | 1 | 12 | 6 | 10 | 72 | 88 | |
| 0 | 2 | 16 | 4 | 6 | 74 | 84 | |
| 0 | 3 | 9 | 2 | 9 | 80 | 91 | 86.0 (±2.0)*** |
| 60 | 1 | 4 | 3 | 6 | 87 | 96 | |
| 60 | 2 | 9 | 9 | 11 | 71 | 91 | |
| 60 | 3 | 9 | 4 | 0 | 87 | 91 | 92.7 (±1.7) |

*Pollen incubated in sucrose/boric acid medium overnight (~16 hr) at 25° C.
**Rating: 1 = not germinated; 2 = pollen tube length < 2x pollen diameter; 3 = pollen tube length 2x-4x pollen diameter; 4 = pollen tube length ≧ 4X pollen diameter.
***standard error of the mean of three replicates.

TABLE 5
Average length of strawberry pollen tube* after
UV-C (254 nm) exposure, followed by 4 hr dark.

| UV exposure | | Pollen tube length | Average pollen tube |
|---|---|---|---|
| (sec) | Replicate | (μm) | length (μm)/treatment |
| 0 | 1 | 218.8 (±3.8)** | |
| 0 | 2 | 206.7 (±15.0) | |
| 0 | 3 | 136.1 (±10.1) | 187.2 (±25.8) |
| 60 | 1 | 178.8 (±10.4) | |
| 60 | 2 | 200.0 (±13.3) | |
| 60 | 3 | 155.7 (±9.6) | 178.2 (±12.8) |

*Pollen incubated in pollen germination solution overnight (~16 hr) at 25° C.
**Standard error of the mean for measurements of 100 pollen tubes.

Flowers were emasculated by removing the anthers from the filaments of newly opened flowers on plants maintained in high tunnel culture. The anthers and pollen were collected as stated above. Emasculated flowers were marked and covered with brown paper bags to prevent natural pollination. The pollen was exposed to UV-C (254 nm) treatment and kept in the dark for four hours. Untreated control pollen and UV-C exposed pollen was spread on the stigmas of separate emasculated flowers using a sterile glass rod. Flowers were then covered with the brown paper bags again. The flowers were detached after 24 hours and brought into the laboratory for microscopic observation. The styles and attached ovaries were carefully separated from the flower receptacle and stained with 0.1% aniline blue in phosphate buffer pH 7.4 for at least 10 min. A cover slip was placed on top and gently pressed with a pencil eraser to facilitate stain penetration by splitting of the style. Specimens were observed under UV illumination using the Zeiss Axiophot microscope and the images of the growing pollen tube were captured with an Olympus DL71 microscope digital camera.

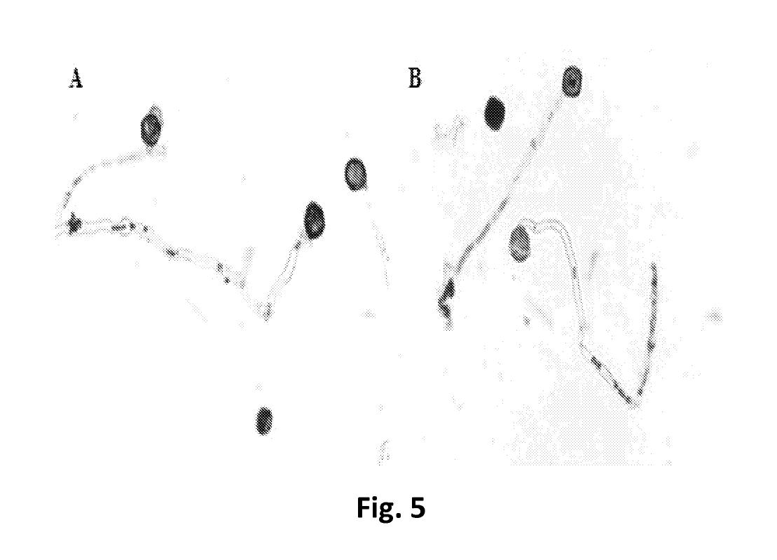

Pollination of strawberry flowers with pollen irradiated for 60 sec and kept in dark for 4 hr resulted in massive germination of pollen on the stigma and rapid tube growth through the style all the way to ovary (FIG. 6).

### Example 4

### Infectivity of Irradiated Conidia

Irradiation of dry conidia of B. cinerea was conducted similar to pollen irradiation, described above, except that the conidia were deposited on the plates in a very thin layer by gently shaking the spore collector tube. After irradiation treatments the conidia were collected in sterile tap water (STW), and concentration was adjusted to 1×10⁴ conidia/mL with hemacytometer before plating or fruit inoculation. The experiments were repeated two times.

Irradiation of dry conidia for 60 sec reduced the number of viable conidia from 89 to 20 CFU/plate (77.5% reduction) (Table 6).

TABLE 6
Viability and infectivity of B. cinerea conidia after
UV-C (254 nm) exposure of dry conidia and 4 hr dark incubation
period before plating and fruit inoculation

| | | | |
|---|---|---|---|
| UV | Colony | Reduction in | Infection of |
| exposure | count | viability (%) | apple wounds* |

| | | | | |
|---|---|---|---|---|
| 0 | sec | 89 | — | + |
| 60 | sec | 20 | 77.5 | − |
| 2 | min | 18 | 79.8 | − |
| 4 | min | 5 | 94.4 | − |

*Each wound was inoculated with 25 μL of 10⁴ conidia/mL suspension.

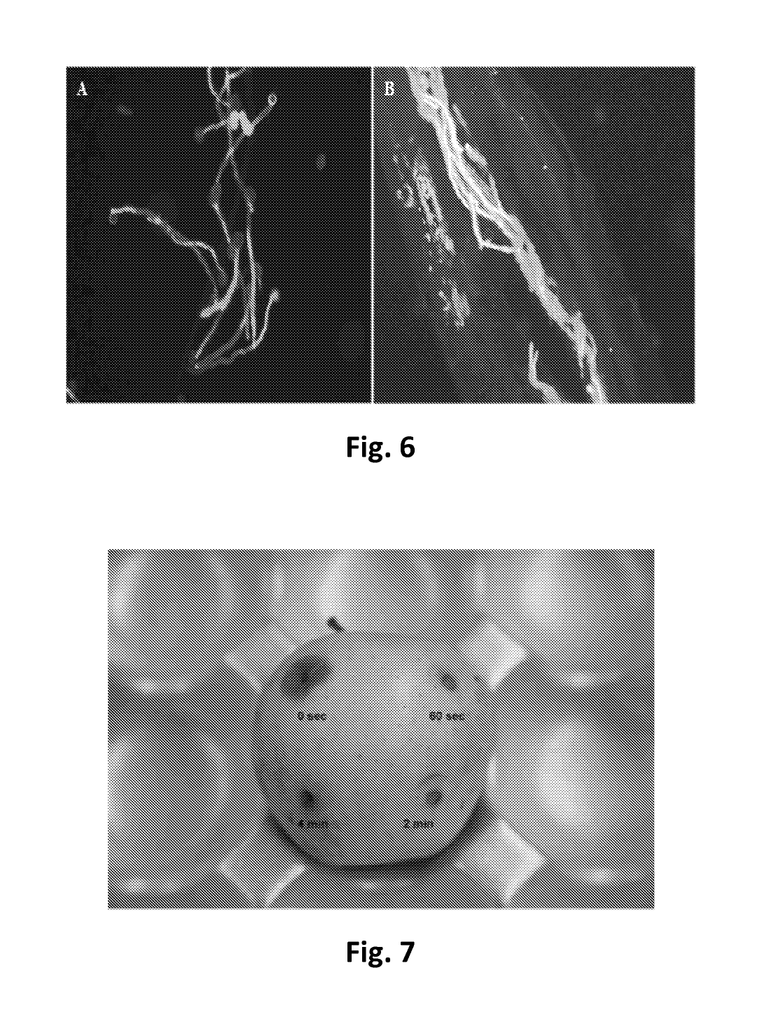

To determine if viable UV-C exposed conidia of B. cinerea were still infective, we used an apple model system. Wounds of mature apples are very susceptible to B. cinerea infection and the resulting fruit decay is easy to see and easily distinguished from decays caused by other pathogens. Mature ‘Golden Delicious’ apples were surface sterilized with 70% ethanol and allowed to dry under a transfer hood to prevent contamination. Apples were wounded in four places with cylindrical tool (3-mm dia. and 3-mm deep) and the cut tissue was removed. Each wound was inoculated with 25 μL (1×10⁴ conidia/mL) suspension containing conidia exposed to different levels of UV-C irradiation. Inoculated fruit were placed in closed boxes and incubated at 22° C. for 5 days. There were four replications for each treatment. Although some conidia were still viable after UV-C irradiation, they did not infect apple wounds (FIG. 7). The effect of UV-C on viability and infectivity may vary depending on the organism.

### Example 5

### Recovery of Antagonist Biocontrol Microorganisms from Strawberry Flowers

There is a growing demand for alternatives to synthetic fungicides for controlling fruit decays in the field and after harvest. Several yeasts and bacteria naturally occurring on fruit have been found to effectively control fruit decays and are available as commercial products for treating fruits. The main prerequisite for the yeast or bacterial biocontrol agents (antagonists) to be effective is their ability to survive and colonize the plant parts that they must protect. Populations of the most effective antagonists increase many fold after application to plants in a relatively short period of time. Examples of some of our most effective antagonists include bacteria Pseudomonas syringae and Serratia grimesii and yeasts Rhodotorula phylloplana, Sporidiobolus pararoseus, Cryptococcus VKMY2958, Metschnikowia pulcherrima and Aureobasidium pullulans.

The two highly effective biocontrol yeasts, antagonist cultures Metschnikowia pulcherrima (FMB-24H-2) and Aureobasidium pullulans (ST1-C9), were grown in 250 mL flasks with 50 mL of nutrient yeast dextrose broth (NYDB) medium overnight at 28° C. on rotary shaker at 250 rpm. The cultures were harvested by centrifugation (7,000 rpm, 4° C., 10 min) and the antagonists were resuspended in STW to make a stock suspension. The antagonist's concentration was adjusted to 80% T (turbidity) at 420 nm using a spectrophotometer.

Strawberry flowers at similar developmental stage were collected with stems and placed in small culture tubes with water on a test tube rack. Using an atomizer, the antagonist suspensions were misted onto strawberry flowers until small droplets were visible. The flowers were covered with a plastic box and allow to air dry before first recovery (time 0) and subsequent incubation at 22° C.

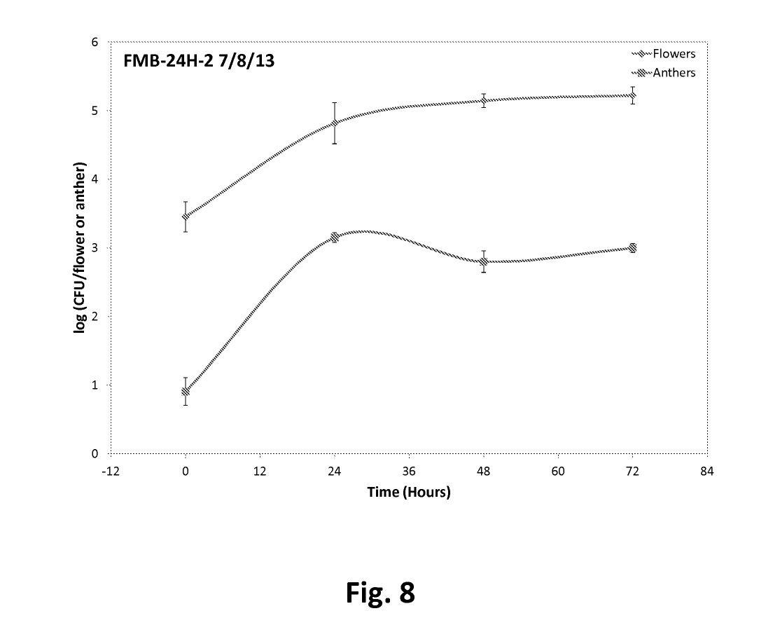

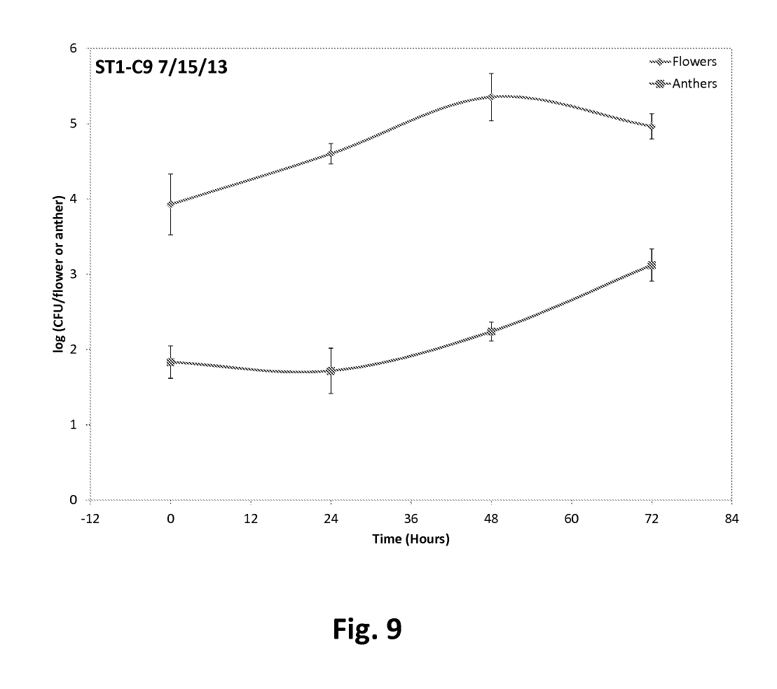

At each recovery time, five flowers were randomly selected from antagonist-treated plants. All anthers on each flower were removed and 10 anthers from each flower were placed in a stomacher blender bag with 5 mL SDW. Each emasculated flower was placed in a separate stomacher bag with 5 mL SDW. The bags were placed in a stomacher blender and blended for 120 sec at normal speed. The resulting suspensions were passed through syringes with glass wool, serially diluted (1:10 dilution) and plated on nutrient yeast dextrose agar (NYDA) medium plates amended with 100 ppm streptomycin (to prevent bacterial growth) using spiral plater (Autoplater 4000, Spiral Biotech, Inc., Norwood, Mass.) and incubated at 24° C. for up to 48 hr until no new colonies appeared. The colonies were counted using the QCount Spiral Biotech plate reader (Spiral Biotech) and the concentrations were determined with the SGE (Spiral Gradient Endpoint) software (Spiral Biotech). Recoveries of M. pulchernma (FMB-24H-2) and A. pullulans (ST1-C9) populations are shown in FIG. 8 and FIG. 9, respectively.

### Example 6

### Treatment Apparatus

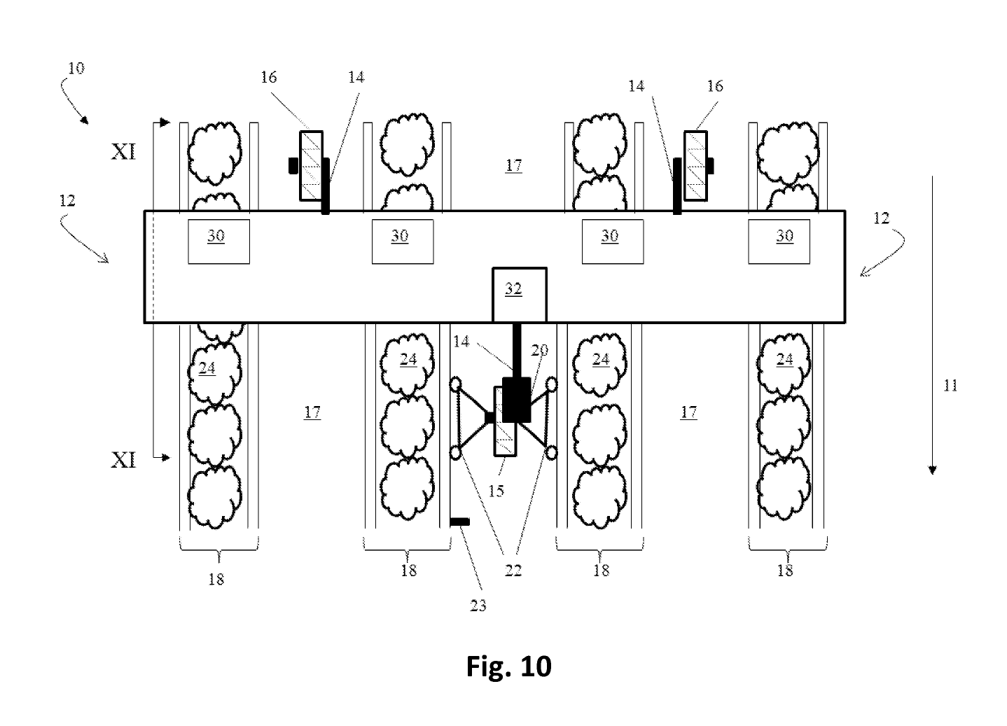

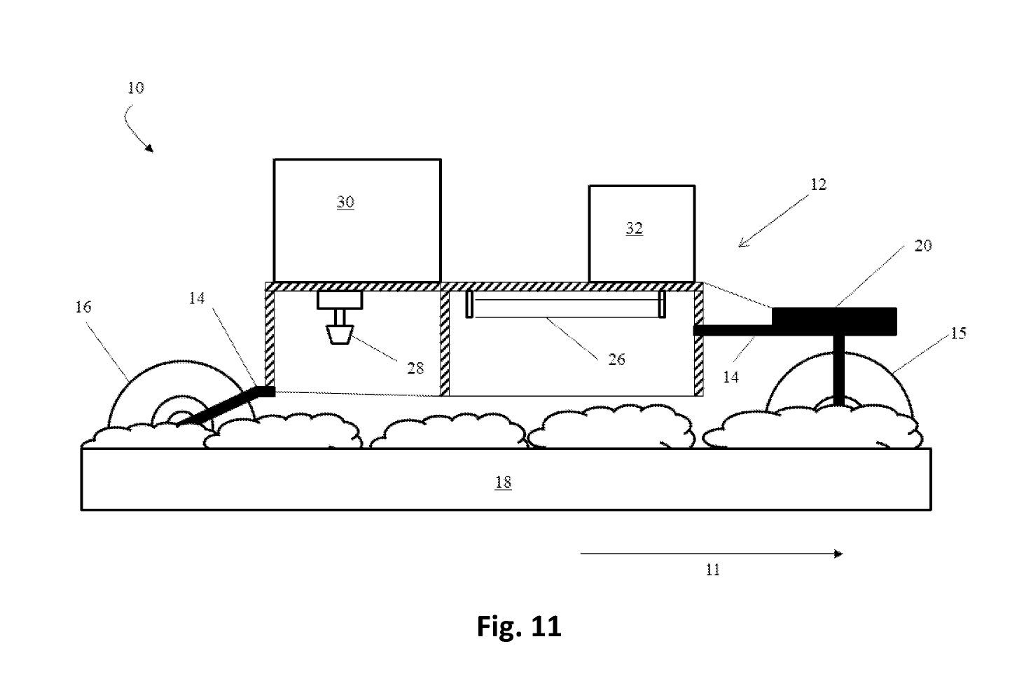

As best shown in FIGS. 10 and 11, in the preferred embodiment, the treatment apparatus 10 comprises a mobile tricycle-type vehicle. FIG. 10 is a top view of the apparatus 10, and FIG. 11 is a sectional view of the treatment module 12 along the section line XI shown in FIG. 10. As shown in FIGS. 10 and 11, in the preferred embodiment, the treatment module 12 moves over elevated plant beds 18 in the direction of the arrow 11 and thereby treats the plants 24.

Specifically, as best shown in FIGS. 10 and 11, the treatment apparatus 10 comprises a treatment module 12 that is mounted on a chassis 14 that is supported by a front wheel 15 and trailing back wheels 16. The wheels 15, 16 are positioned in the aisles 17 between elevated plant beds 18. A steering and drive mechanism 20 is mounted adjacent to the front wheel 15. A pair of roller assemblies 22 extends laterally from the front wheel 15 area to a vertical wall portion of the elevated plant bed 18. The roller assemblies 22 (among other things) maintain the front wheel 15 centered in the aisle 17 and correspondingly maintains the treatment module 12 in the correct position to treat the plants 24. In alternative embodiments associated with non-elevated plant beds, directional control may be maintained by a laser guidance system or by any other means known in the mechanical arts.

As best shown in FIG. 11, the treatment module 12 generally comprises a UV light array assembly 26 and a spray mechanism 28 surrounded by associated shielding and support structures. In the preferred embodiment the light array emits UV C light. The spray mechanism 28 may include an electrical pump and/or liquid pressurization means and at least one spray nozzle. The spray mechanism 28 is connected to and supplied by a spray tank 30.

As the apparatus is propelled in the direction of the arrow 11, the plants and plant beds are irradiated by the UV light array 24 and sprayed with a biocontrol agent by the spray mechanism 28. In the preferred embodiment, biocontrol agents comprise living microorganisms that are not harmful and may be beneficial to the plant. As discussed in other sections of this disclosure, the presence of the biocontrol agents substantially prevents surfaces of a plant from becoming re-colonized by pathogenic (as defined herein) microorganisms after the plant is irradiated. The treatment module 12 is structured so that a UV light array 26 and corresponding spray mechanism 28 are arranged in tandem and are sequentially moved over the plants 24 in each of the elevated beds 18.

The functions of the treatment apparatus 10 are controlled by a programmable electronic controller 32. In the preferred embodiment, the controller 32 coordinates electronic signals both to and from the UV light array assembly 26, the spray mechanism 28, and the steering and drive mechanism 20. The controller 32 may also receive signals from the roller assemblies 22 via paddles 23 or other structures extending from the vertical walls of the elevated beds 18 so that when (for example) the roller assembly 22 encounters a paddle 23 (for example a stop), an electronic signal is sent to the controller 32 indicating that the apparatus 10 is at or near the end of the elevated bed 18 and therefore treatment should be terminated. Alternatively, paddles 23 (that are not stops) may signal to the controller to change speed or modify the treatment protocol via the light array assembly 26, spray mechanism 28, or steering and drive mechanism 20. Essentially, the controller 32 controller actively controls the speed and treatment functions of the treatment apparatus 10.

In operation, as shown in FIGS. 10 and 11, treatment begins as the apparatus 10 moves forward in the direction of the arrow 11. As the light array 26 passes over the plants 24, the plants 24 are irradiated by UV light per the protocol described in other sections of this disclosure. Immediately following irradiation, the spray mechanism 28 sprays the plants 24 with a biocontrol agent per the protocol described in other sections of this disclosure. During the treatment process, a controller 32 monitors and controls the treatment, and subsequently terminates the treatment at the conclusion of the prescribed protocol. Optionally, the controller 32 may reset/reposition the apparatus 10 for subsequent treatment operations.

In alternative embodiments, the form and function of the treatment apparatus 10 may vary significantly. For example, the apparatus 10 may have more or fewer than three wheels 15, 16. In further embodiments (particularly with non-elevated plant beds), the apparatus 10 may use a laser guidance system so that the steering and propelling means guides the apparatus 10 along a laser trajectory defined by an operator. In some applications the treatment may take place in a completely dark environment so that guidance of the apparatus 10 should not depend on visual cues and high degrees of automation are desirable.

Additionally, the apparatus 10 may not include a drive and steering mechanism 20 so that the apparatus 10 is pulled in the direction of the arrow 11 by a winch system or a or the like. In further alternative embodiments the wheels 15, 16 may be optionally replaced by skids or other support means. The apparatus may also be towed by a utility vehicle such as a tractor with a “crawler” type transmission. Although the preferred embodiment spans four rows 18 of elevated living plants 24, in alternative embodiments the treatment apparatus 10 may span greater or fewer than four rows of plants 24 and (as discussed supra) the plant beds 18 may not be elevated or may even be recessed relative to ground level.

Further, the controller 32 may or may not be physically positioned on the treatment module 12 so that the controller 32 communicates with the apparatus 10 wirelessly. Although the irradiating light array 26 and the spray dispenser 28 are shown schematically in FIG. 11, in alternative embodiments the lights 26 may have a variety of forms and the spray dispenser may comprise a plurality of nozzles positioned in different configurations.

The apparatus 10 described herein may be modified in multiple ways and applied in various technological applications and the individual components may be modified and defined, as required, to achieve a desired result. Although the materials of construction are not described, they may include a variety of compositions consistent with the function described herein. Such variations are not to be regarded as a departure from the spirit and scope of this disclosure, and all such modifications as would be obvious to one skilled in the art are assumed to be within the scope of the invention described herein.

### Example 7

### High Tunnel UV-C Treatment

Strawberry plants (cv. Albion) in 6-inch pots were established in four rows of raised beds in high tunnel culture. All runners, fruit, and flowers were removed from the study plants prior to start of UV-C treatment. Each row had a designated UV-C irradiation exposure duration (0, 30, or 60 sec of UV-C irradiation) and divided into 5 plots (replicate R1-R5). Five additional plots at the ends of 2 rows were designated for fungicide treatment (F1-F5).

Each treatment day (Monday and Thursday) the apparatus was turned on at the end of the day and the timer was set to start the irradiation treatments at 11:00 pm. The self-propelled apparatus travelled down the rows (plot 1 to 5) delivering the designated dose of UV-C irradiation. After irradiating the last plot (plot 5), the apparatus remained idle until next morning when it was pushed back to the end of the high tunnel to be ready for the next treatment.

Harvests began 3 weeks after the first UV-C treatment and were performed each Monday and Thursday. Fruit were visually assessed for ripeness based on the color (mostly red). Ripe fruit were cut from the plant leaving approximately′-inch stem attached and placed in labeled weigh boats. Both healthy and diseased fruit were harvested. Fruit from each plot (treatment replicate) were counted and weighed. Each fruit was then assessed as diseased or sound (healthy), well-shaped or deformed, and weighing more or less than 8 g.

A total of 13 harvests were made over the 7-week harvest period. Total fruit count and yield weights were compiled for each replicate and treatment, and averages per plant were calculated along with standard error of the means (Table 7).

All publications and patents mentioned in this specification are herein incorporated by reference to the same extent as if each individual publication or patent was specifically and individually indicated to be incorporated by reference.

The foregoing description and certain representative embodiments and details of the invention have been presented for purposes of illustration and description of the invention. It is not intended to be exhaustive or to limit the invention to the precise forms disclosed. It will be apparent to practitioners skilled in this art that modifications and variations may be made therein without departing from the scope of the invention.

TABLE 7
Fruit count and weight from harvested* strawberry plants grown in
high tunnels and exposed to UV-C** two times a week for 7 weeks.

| | Fruit weight (g) | | | Count | | Avg. sound |
|---|---|---|---|---|---|---|
| Treatment | Sound | Well-shaped | Deformed | Total fruit | Deformed | per plant (g) |
| CK | 1458.7 ± 94.4 | 1299.0 ± 47.9 | 198.7 ± 31.8 | 78.6 ± 4.1 | 15.2 ± 2.1 | 364.7 ± 23.6 |
| 30 sec UV | 1371.7 ± 86.6 | 1211.8 ± 50.5 | 152.4 ± 35.5 | 67.4 ± 3.6 | 10.8 ± 2.7 | 342.9 ± 21.6 |
| 60A sec UV | 1543.5 ± 73.0 | 1423.3 ± 80.1 | 133.6 ± 18.8 | 84.6 ± 2.5 | 11.2 ± 1.3 | 385.9 ± 18.3 |
| 6B0 sec UV | 1311.1 ± 53.0 | 1224.8 ± 47.3 | 102.9 ± 17.2 | 68.2 ± 3.5 | 7.4 ± 1.2 | 327.8 ± 13.2 |
| Fungicide | 1211.5 ± 134.0 | 1041.4 ± 108.6 | 173.6 ± 28.1 | 65.0 ± 5.7 | 11.8 ± 1.7 | 302.9 ± 33.5 |

*Fruit were harvested in weeks 7 through 10.
**UV-C exposure: 2 × per week for 10 weeks

We claim:

1. A treatment method for reducing, total amount of one or more pathogenic populations on a living plant resulting in reduction of diseases they cause; said method comprising:
(a) exposing a live plant, or a part thereof, to a dosage of UV-C having a peak wavelength of about 254 nm for a period of time of about 15, 30 or 60 seconds depending on the sensitivity of said pathogens;
(b) allowing said plant to remain in continuous darkness for a period of at least 2 hours immediately following UV-C irradiation;
(c) administering an antagonist biocontrol microorganism subsequent to UV-C irradiation; and
(d) observing a reduction or absence of the infectivity of the fungi/pathogens.

2. The treatment method according to claim 1 wherein during a period of exposure to UV-C, the dosage of UV-C irradiation is about 0.001236 J/cm² of plant aerial surface.

3. The treatment method according to claim 1, wherein the growth and/or infectivity of one or more pathogenic populations is reduced by at least 50% compared to an equivalent control that has not been contacted with said treatment method.

4. The treatment method according to claim 1, wherein said one or more pathogens comprises one or more of a fungus or bacterium.

5. The treatment method according to claim 1, wherein said one or more pathogens comprise one or more of: Botrytis, Colletotrichum, Podosphaera or Erisiphe or other pathogens residing on aerial plant surfaces.

6. The treatment method of claim 5, wherein said other pathogens residing on aerial plant surfaces are one or more of: Leveillula taurica (previously Erysiphe taurica), Oidium neolycopersicum, Podosphaera xanthii (previously Sphaerotheca fuliginea), Oidium dianthi, Podosphaera pannosa var. rosae (previously Sphaerotheca pannosa var. rosae), Podosphaera fusca, Microsphaera syringae and Phyllactinia corylea.

7. The treatment method according to claim 1, wherein the plant is a vegetable or ornamental plant species or a fruit-bearing species.

8. The treatment method according to claim 1, wherein the plant is grown in greenhouse, tunnel, field production systems, and/or in other controlled environments.

9. The treatment method according to claim 1, wherein said exposing step is carried out during more than one developmental stage of said plant.

10. The treatment method according to claim 9, wherein said exposing step for flowering plants is carried out during the blooming stage of said plant.

11. The treatment method of claim 1 wherein at least one of the parts is a flower or an aerial part.

12. The treatment method of claim 1 wherein the antagonist biocontrol microorganism administered subsequent to UV-C irradiation and said period of darkness is any one of: Pseudomonas syringa, Serratia grimesii, Rhodotorula phylloplana, Sporidiobolus pararoseus, Cryptococcus VKMY2958, Metschnikowia pulchernma and Aureobasidium pullulans.

13. A treatment method of claim 1 wherein organisms of said pathogenic population may be still viable after treatment but are not infective as determined by an assay which measures infectivity.

14. A plant treatment apparatus comprising an irradiation array in combination with a biocontrol dispenser, the treatment apparatus being structured so that as the treatment apparatus moves along a plant bed, a targeted living plant is irradiated by the irradiation array, and immediately thereafter treated with a biocontrol agent by the biocontrol dispenser so that pathogenic microorganisms are killed by the irradiation and substantially prevented from colonizing surfaces of the plant by the biocontrol agent.

15. The apparatus of claim 14 wherein the targeted plant is irradiated by a UV light irradiation array.

16. The apparatus of claim 15 wherein the UV light irradiation array comprises a UV C light irradiation array.

17. The apparatus of claim 14 wherein the biocontrol dispenser sprays the targeted plant with a biocontrol agent comprising living microorganisms.

18. The apparatus of claim 14 wherein irradiation apparatus and the biocontrol dispenser are disposed in a single housing comprising a treatment module, the treatment module being supported by a wheeled chassis so that the apparatus rolls along the plant bed.

19. The apparatus of claim 18 wherein the wheeled chassis has a tricycle configuration so that the treatment module spans multiple rows of plants.

20. The apparatus of claim 18 wherein the apparatus further comprises a steering mechanism and driving mechanism so that the apparatus is self propelled.

21. The apparatus of claim 20 wherein the steering and driving mechanism is configured so that the driving mechanism drives a front wheel.

22. The apparatus of claim 21 wherein the steering mechanism controls the front wheel.

23. The apparatus of claim 22 wherein the plant bed comprises an elevated plant bed, the elevated plant bed comprising vertical side walls that define the plant bed.

24. The apparatus of claim 23 further comprising roller assemblies that extend from the vertical side walls to an area adjacent to the front wheel, and thereby control steering of the front wheel.

25. The apparatus of claim 22 further comprising an electronic controller, the controller controlling the steering and drive mechanism as well as the irradiation array and biocontrol dispenser so that the controller effectively controls the apparatus and thereby defines a treatment protocol.

26. The apparatus of claim 14 wherein the apparatus further comprises an electronic controller that is in communication with the irradiation array and the biocontrol dispenser.

27. The apparatus of claim 26 wherein the controller is programmable so that the controller defines a treatment protocol administered by the apparatus.

28. A method of treating at least one plant bed comprising multiple targeted plants, the method comprising:
(a) providing the apparatus of claim 26;
(b) moving the apparatus along the plant bed so that the targeted plants are treated in accordance with the treatment protocol.
